(19) United States
(12) Patent Application Publication
(10) Pub. No.: US 2009/0275667 A1
(43) Pub. Date: Nov. 5, 2009

(54) USE OF ALLICIN AS PRESERVATIVE, AS DISINFECTANT, AS ANTIMICROBIAL OR AS BIOCIDAL AGENT

(75) Inventors: Norman J. Bennett, Rye (GB); Peter D. Josling, Rye (GB)

Correspondence Address: CONNOLLY BOVE LODGE & HUTZ LLP, 1875 EYE STREET, N.W., SUITE 1100, WASHINGTON, DC, 20006, US

(73) Assignee: Stone Island Holdings Ltd, East Sussex (GB)

(21) Appl. No.: 12/465,716

(22) Filed: May 14, 2009

Related U.S. Application Data
(62) Division of application No. 10/551,235, filed on Jul. 11, 2006, now abandoned.

(30) Foreign Application Priority Data
Mar. 27, 2003 (GB) 0307079.4

(51) Int. Cl. A01N 41/00

(52) U.S. Cl. 514/707

(57) ABSTRACT

The present invention provides the use of allicin in (i) the treatment of leishmanimis; (b) as a disinfectant or biocidal treatment of aquatic species; (iii) as an antimicrobial agent for animal feed; (iv) as a preservative agent in foodstuffs; (v) as a water disinfectant or biocide; (vi) as an antiparasitic treatment or antibacterial treatment for bees (apis); or (vii) in the preparation of a medicament for the treatment of Glycopeptide Intermediate Resistant Stapohylococcus aureus.

The present invention relates to allicin.

Allicin, a sulphur compound having the formula:

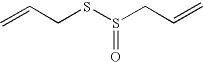

is thought to be the principal active compound giving rise to the numerous therapeutic properties which are claimed for garlic (Allium sativium). In the natural state, garlic does not contain allicin, but a precursor, alliin [(+) S-allyl-L-cysteine sulphoxide]. Alliin is converted into allicin by the action of the enzyme allinase or alliin lyase, also a component of garlic. Alliin and allinase are brought together when garlic cloves are cut or crushed. The following equation represents the synthetic route.

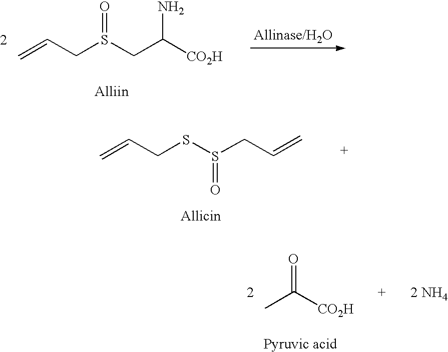

However, allinase is rapidly and irreversibly deactivated by its reaction product, allicin, and is also deactivated in acid conditions such as the stomach. Thus, in practice, the yield of allicin from a clove of garlic falls far short of the theoretical maximum. Indeed, yields are usually of the order of 0.3-0.5%.

WO97/39115 describes a continuous process for the synthesis of allicin by preparing a column containing allinase immobilised on a solid support, passing a solution of alliin through the column and collecting a solution of allicin in the effluent.

Allicin is also prepared by the present applicant in liquid and spray-dried forms and is available in capsules and bulk powder form from Allicin International Limited of Half House, Military Road, Rye, East Sussex, TN31 7NY, United Kingdom, under the trade mark ALLIMAX.

In our co-pending PCT application, WO03/024437, published on 27 Mar. 2003, we describe certain novel therapeutic properties of allicin.

The present invention is based on further investigations into therapeutic properties of allicin.

### SUMMARY OF THE INVENTION

In its broadest sense, the present invention provides the use of allicin in (i) the treatment of leishmaniasis; (ii) as a disinfectant or biocidal treatment of aquatic species; (iii) as an antimicrobial agent for animal feed; (iv) as a preservative agent in foodstuffs; (v) as a water disinfectant or biocide; (vi) as an antiparcistic treatment or antibacterial treatment for bees (apis); or (vii) in the preparation of a medicament for the treatment of Glycopeptide Intermediate Resistant Staphylococcus aureus.

In one aspect, the present invention provides the use of allicin in the treatment of leishmaniasis. The present invention also provides the use of allicin in the preparation of a medicament for treatment of leishmaniasis. Preferably, allicin is present in the medicament at a concentration of about 5000 ppm.

In a second aspect, the present invention provides the use of allicin as a disinfectant or biocidal treatment of aquatic species. The present invention also provides the use of allicin in the preparation of a medicament for disinfection or biocidal treatment of aquatic species. Typically, the aquatic species are fish. This aspect of the present invention is particularly applicable to the fish farming and other aquatic or marine industries.

In a third aspect, the present invention provides the use of allicin as an antimicrobial agent for animal feed. Suitably the animal feed is water feed and allicin is present in an amount of about 500 ppm. In an alternative embodiment, the animal feed is a feedstuff and allicin is present in an amount giving a daily intake of from 1 to 5 mg per animal per day. Suitably, for large animals such as cows or horses, allicin is present in an amount giving a daily intake of from 2.5 to 3 mg per animal per day. For smaller animals such as pigs or goats, allicin is present in an amount giving a daily intake of from 1.5 to 2.4 mg per animal per day.

In a fourth aspect, the present invention provides the use of allicin as a preservative agent in foodstuffs. The present invention also provides a food preservative agent comprising allicin and at least one food-grade excipient. Preferably, the preservative agent comprises allicin in a concentration of up to 500 ppm.

In a fifth aspect, the present invention provides the use of allicin as a water disinfectant or biocide. The present invention also provides a water treatment composition comprising allicin and a food-grade excipient. In particular it provides such a water disinfectant or biocide for use in vegetable washing water, wastewater, stormwater or potable water treatments. Preferably, the water treatment composition comprises allicin in an amount of from 0.5 to 2.0% w/v or w/w, more preferably in an amount of 0.9 to 1.7%.

In a sixth aspect, the present invention provides the use of allicin as antiparasitic and antibacterial treatment for bees (apis). The present invention also provides the use of allicin in the preparation of an antiparasitic treatment for bees. The present invention also provides an antiparasitic treatment for bees comprising allicin and a pharmaceutically acceptable excipient. In particular, this aspect of the present invention provides a treatment against the Varroa mite and the bacteria Melissococcus plutonius (formerly called Streptococcus plutonius) and Paenibacillus larvae subsp. Larva and the fungal brood disease chalkbrood Ascophera apis.

In a seventh aspect, the present invention also provides the use of allicin in the preparation of a medicament for the treatment of Glycopeptide Intermediate Resistant Staphylococcus aureus.

### BRIEF DESCRIPTION OF THE FIGURES

FIG. 1 illustrates a plate assay showing zones of inhibition of Escherichia coli around wells of Allicin solution on a nutrient agar plate. The figure shows, anticlockwise from top right, 100%, 50%, 12.5% and 25% dilutions of the original allicin solution (1.8% w/v).

FIG. 2 illustrates a plate assay showing a zone of inhibition produced by 500 ppm of allicin against a GISA strain.

### DETAILED DESCRIPTION OF THE INVENTION

Suitably for oral administration, or administration as a suppository, pessary or nasal preparation, the pharmaceutically acceptable excipient is a solid composition onto which the allicin or its metabolite is bound. More suitably, the solid composition comprises a bullring agent, such as lactose, microcrystalline cellulose or dicalcium phosphate, preferably cellulose; a thickening agent such as a gum or starch; a disintegrant, such as sodium starch glycolate or cross-linked povidone; a release agent such as magnesium stearate; an emulsifying agent; a surfactant and such sweeteners, fragrances and colorants as may be desired. Most preferably, allicin is bound by a spray drying process and the solid composition comprises a modified starch such as maltodextrin, gum acacia, silica and an emulsifier such as magnesium stearate. WO02/062416 describes an apparatus for dispensing powdered material. It has been found that this apparatus is advantageous in delivery of a composition comprising allicin and a cellulose powder. Accordingly, in a final aspect of the present invention there is provided a composition comprising allicin and a cellulose powder.

Suitably, for topical application, the pharmaceutically acceptable excipient comprises a cream or a soap. The excipient may, alternatively, constitute a lotion, ointment, toothpaste, mouthwash or a hair preparation such as a shampoo, styling gel or conditioner. Such preparations may include a combination of the following as appropriate: surfactants, fragrances, colours, stabilisers, antioxidants, emulsifying agents, thickening agents, waxes, glycerols, fats, suspending agents, de-flocculating agents and antioxidants all of which may or may not be hypo-allergenic. Suitably, a cream excipient comprises white soft paraffin, an emulsifier such as a stearate, suitably magnesium stearate, glycerin, water, yellow soft paraffin and a stabiliser, such as potassium citrate. Most suitably, a cream excipient comprises an aqueous cream, preferably Aqueous Cream BP. Suitably, a soap excipient comprises ether sulphate, cocamide and cocobetaine. Optionally, the excipient may further include fragrances and colorants.

Suitably, for oral, parenteral and topical application, the ratio of allicin to excipient is such as to provide an allicin concentration of between 1 ppm and 2000 ppm, preferably between 50 and 1000 ppm, more preferably between 250 and 500 ppm.

The above and other aspects of the present invention will now be described in further detail, by way of example only.

### 1. The Use of Allicin in the Treatment of Leishmaniasis

Leishmaniasis is a disease common in the tropics and sub-tropics caused by parasitic protozoans of the genus Leishmania which are transmitted by the bite of sandflies. There are two principal forms of the disease—visceral leishmaniasis in which the cells of various internal organs are affected and cutaneous leishmaniasis which affects the tissues of the skin. This latter form itself has several different forms depending on the region in which it occurs and the protozooal species involved. Countries such as Panama, Honduras, the Amazon, South Central America and Asia are the areas where leishmaniasis is the most common. In Asia for example, it is common in the form of an oriental sore and can be seen as a major third world problem. Leishmaniasis is a disease of the skin and mucous membranes resulting in ulcerating lesions found on the arms and legs. The infection may also spread to the mucous membranes of the nose and mouth causing serious destruction of the tissues. Standard treatment is normally with drugs containing antimony but these are generally not readily available or well tolerated.

A form of leishmaniasis of the skin caused by the parasite leishmania tropica mexicana is also known as Chiclero's ulcer. The disease occurs in Panama, Honduras and the Amazon and primarily affects men who visit the forests to collect chicle (gum). This condition takes the form of an ulcerating lesion on the ear lobe and although the sore usually heals spontaneously within 6 months this can however cause a great deal of discomfort.

Confirmatory in vitro tests at the University of East London using allicin at a concentration of 5.0 gm per litre has killed the protozoal parasite associated with Leishmaniasis. Taken with extrapolation of the results from the laboratory studies described in PCT/GB2002/004309, we believe that allicin at a concentration of 5000 ppm has efficacy as an antiprotozoal agent.

### 2. The Use of Allicin as a Disinfectantbiocide in Fish Farming and Other Aquatic or Marine Industries.

We have demonstrated that allicin can be used in fish farming and other aquatic industries to kill bacteria, parasites and fungi. Allicin can be used as an antimicrobial (including antibacterial, antiviral, antifungal and anti protozoal) preparation comprising allicin (and its metabolites, including DADS (Diallyldisulphide), DATS (Diallyltrisulphide), ajoene, allitridium and vinyldithiins).

Based on the test results from our laboratory studies on MRSA (30 strains), E. coli, E. faecalis, Candida albicans, Pseudomonas aeruginosa, Salmonella typhimurium, Streptococcus pyogenes, B. subtilis, Serratia marcecens. etc, we believe that the results show that allicin can be used as an agent against bacteria and fungi.

Based on the results of our laboratory tests on lice (Pediculus humanus) contained in WO03/024437, we believe that allicin will destroy the parasites associated with fish farming and other aquatic or marine industries.

### 3. The Use of Allicin as an Antimicrobial Agent in Animal Feed.

Allicin can be used as an antimicrobial agent in animal feed to promote growth in animals, prevent disease in animals and prevent the transmission of disease (including food poisoning) to humans. The antimicrobial (including antibacterial, antiviral, antifungal and anti protozoal) preparation comprises allicin (and its metabolites, including DADS (Diallyldisulphide), DATS (Diallyltrisulphide), ajoene, allitridium and vinyldithiins). Animals (for example, chickens, pigs, goats and cows) can pick up bacteria and pass these through the food chain to the human population. Conventional animal feedstuff and additives (including antibiotics) are used to prevent and treat disease in animals. Forthcoming European legislation suggests that the use of antibiotics may be banned or, at best, restricted.

### Tests and Doses

Our earlier application, WO03/024437, describes laboratory tests which show that allicin can kill E. coli, Listeria, E. faecalis and other bacteria associated with animal diseases at a range of concentrations of up to 500 ppm. By dosing the water feed channels of chickens with allicin at a concentration of 500 ppm, allicin can be used as an antimicrobial preventative product. By dosing the feedstuff of animals such as pigs and goats with 1.5 mg to 2.4 mg of allicin per day, allicin can be used as an antimicrobial preventative product. By dosing the feedstuff of larger animals such as cows and horses with 2.5 mg to 3.0 mg of allicin per day allicin can be used as an antimicrobial preventative product.

An in vivo trial has also been conducted in which two adjacent sheds each of 10,000 chickens were compared. Each shed was supplied with 1000 litres of water per day. For 7 days, the water for one shed had 1.5 litres of allicin solution (1000 ppm) added to the water supply, with 1 litre of allicin solution (1000 ppm) being added per day for a further 3 days. In the control shed, no allicin solution was added.

After just a few days, an improvement in the appearance of the health of the chickens in the allicin-treated shed was noted. For example, the combs of the chickens appeared redder and there was an increase of 2 percent in egg production. The vitality of the chickens improved. In contrast, in the control barn, E. coli infection was observed. Following completion of the trial, the livers of several of the birds were examined. The livers of the control chickens showed evidence of E. coli infection whereas those of the allicin-treated birds did not. The allicin-treated birds exhibited improved metabolism and anti-microbic function. The allicin-treated chickens also showed improved resistance to chicken bloodlice.

In vivo tests, showed the following results against 4 common chicken bacteria gave the following Zones of Inhibition results at 1000 ppm and 166 ppm (1:6 dilution):

| | 1000 ppm | 166 ppm |
|---|---|---|
| E. coli | 17 mm | 0 mm |
| Staph. Aureus | 32 mm | 0 mm |
| Bordetella | 36 mm | 18 mm |
| Salm. Enteriditis | 22 mm | 0 mm |

### 4. The Use of Allicin as a Preservative Agent in Food Processing.

Allicin can be used in food/meat processing to prevent the growth of bacteria that could cause and spread disease (including food poisoning) in humans, by means of an antimicrobial (including antibacterial, antiviral, antifungal and anti protozoal) preparations of allicin (and its metabolites, including DADS (Diallyldisulphide), DATS (Diallyltrisulphide), ajoene, allitridium and vinylditbiins);

A range of concentrations of liquid allicin (0 ppm to 500 ppm) was applied to 10 kg samples of hamburger meat to determine how long bacterial growth could be prevented. These tests were compared to the normal use of existing preservatives (including nitrates and phosphates).

To test for bacterial growth, small samples of meat were cut from the test piece of meat and, using standard methods of analysis, were checked for E. coli and Salmonella growth.

### Results

Allicin liquid with a concentration of 250 ppm prevented bacterial overgrowth for up to 7 days.

Allicin liquid with a concentration of 375 ppm prevented bacterial overgrowth for up to 10 days.

Allicin liquid with a concentration of 500 ppm prevented bacterial overgrowth for up to 14 days.

A control sample of meat with no preservative or allicin showed strong bacterial growth after a few days.

Existing preservatives applied according to permitted normal practice prevented bacterial overgrowth for up to 7 days only.

The study demonstrated that allicin can be used as a preservative in food/meat processing. Standard methods of analysis demonstrated prevention of growth of E. coli and Salmonella at allicin concentrations of 250 ppm (equivalent to 0.0250% w/v). Further evidence to demonstrate the preservative effect of allicin can be extrapolated from the test results of our laboratory studies on MRSA (30 strains), E. coli, E. faecalis, F. streptococcus, Candida albicans, Pseudomonas aeruginosa, Salmonella typhimurium, Streptococcus pyogenes, B. subtfis, Serratia marcecens, Listeria monocytogenes contained in PCT/GB2002/004309, confirming that allicin can be used as a preservative in food/meat preservation.

### 5. The Use of Allicin as a Disinfectantbiocide in Vegetable Washing Wastewater (Including Stormwater) Treatment and Drinking Water Treatment.

Allicin can be used to displace or supplement existing harmful forms of disinfectant/biocide such as chlorine, sodium hypochlorite, ozone and per-acetic acid all of which can have an adverse effect on the environment. UV radiation is also used for disinfection but power and general running costs are high. Laboratory studies have been conducted on our behalf using allicin on aqueous suspensions of bacterial species commonly used as indicators of the effectiveness of water and wastewater disinfection. To this effect, identified isolates from the faecal coliform and streptococcus groups, namely, Escherichia coli (NCTC 8156) and Enterococcus hirae (University of Brighton isolate) were used in all experiments. An aqueous solution of allicin with a nominal concentration of allicin of 1.8 g per litre ie a 0.18% solution was used.

Stock suspensions of Escherichia coli (NCTC 8156) and Enterococcus hirae (University of Brighton isolate) were cultivated in Nutrient Broth No. 2 from freeze dried isolates. Prior to each experiment, serial dilutions of the suspensions were enumerated by the spread plate method on Nutrient Agar and subsequent incubation at 37 deg C.

### The KELSEY-SYKES Test

Initial experimental procedures were based on the methods set out in an established UK protocol (BS 6905: 1987). The Kelsy-Sykes methodology was developed as a guide to the concentrations of disinfectants which may be recommended for use under “dirty” (wastewater/sewage) conditions. It is therefore a suitable means of establishing the effectiveness of a disinfectant against a wastewater containing particulate and dissolved contaminants in addition to micro-organisms.

The basic Kelsy-Sykes test is used to establish the concentration of disinfectant and the contact time at which 3 out of 5 tubes demonstrate no growth of the test organism. It is not designed to demonstrate the percentage kill of the test organism under any set of conditions. Therefore, with regard to wastewater/sewage considerations, the protocol was adapted. Under the revised methodology, a sample was taken from the bacterial suspension/biocide mix after the prescribed contact time and plated out onto solid media so that a colony count could be made (see Tables 1 and 2).

### Agar Inhibition Tests

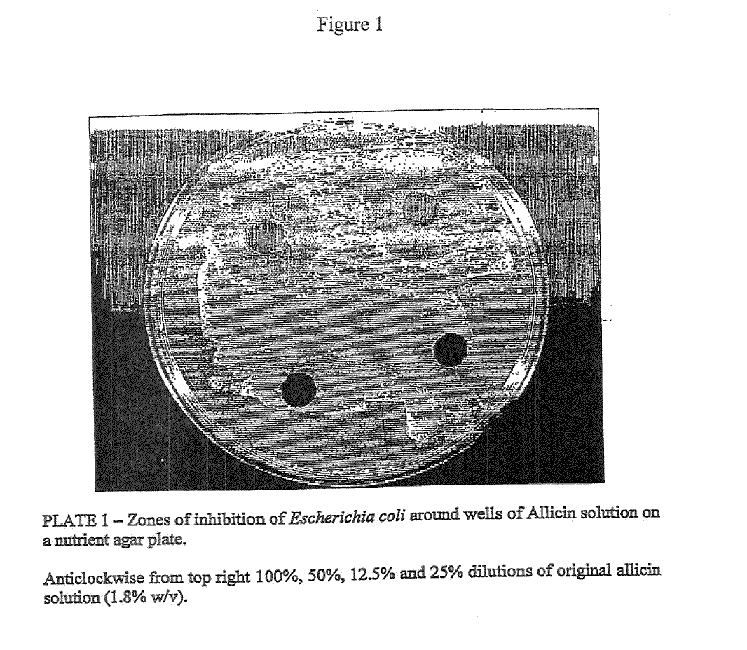

This method was used to show the bactericidal effect of allicin and to study the zone of inhibition produced by the allicin solution on confluent growth of the test organisms on nutrient agar plates. Allicin solution concentrations of 100%, 50%, 25% and 12.5% (in sterile distilled water) were pipetted into wells cored into Nutrient Agar plates on which E. coli isolate had been spread and cultured for 24 hours at 37 deg C. All plates were incubated for a further 24 hours at the same temperature and the zones of inhibition examined (see FIG. 1).

### Results

TABLE 1
percentage reduction in colony forming units of E. coli and
Ent. hirae as a result of contact with allicin solutions in a
modified Kelsey-Sykes test.

| | Percentage reduction in colony forming units | | | | | |
|---|---|---|---|---|---|---|
| Allicin conc. | Escherichia coli | | | Enterococcus hirae | | |
| % (w/v) | 10 mins | 20 mins | 30 mins | 10 mins | 20 mins | 30 mins |
| 0.9 | CG | CG | CG | CG | CG | CG |
| 1.08 | CG | CG | 94 | CG | CG | 77 |
| 1.26 | CG | CG | 97 | CG | CG | 91 |
| 1.4 | CG | CG | 89 | CG | CG | 87 |
| 1.62 | CG | CG | 83 | CG | CG | 89 |

KEY:
CG = Confluent growth

TABLE 2
Numbers of colony forming units of E. coli and Ent. hirae killed
as a result of contact with allicin solutions in a modified
Kelsey-Sykes test.

| | Numbers of colony forming units killed | | | | | |
|---|---|---|---|---|---|---|
| | Escherichia coli | | | Enterococcus hirae | | |
| Allicin conc. | 10 | 20 | 30 | 10 | 20 | 30 |
| % (w/v) | mins | mins | mins | mins | mins | mins |
| 0.9 | CG | CG | CG | CG | CG | CG |
| 1.08 | CG | CG | 4.136 × 10E9 | CG | CG | 1.85 × 10E9 |
| 1.26 | CG | CG | 3.2 × 10E9 | CG | CG | 1.64 × 10E9 |
| 1.44 | CG | CG | 1.96 × 10E9 | CG | CG | 1.04 × 10E9 |
| 1.62 | CG | CG | 9.13 × 10E8 | CG | CG | 5.34 × 10E8 |

KEY:
CG = Confluent growth

The study has demonstrated the bactericidal effect of allicin against bacteria commonly used as indicators of disinfection in water treatment. Simple tests on agar plates demonstrated inhibition of E. coli and Ent. Hirae at allicin concentrations as low as 0.225 g/l (equivalent to 0.0225% w/v). Further evidence to demonstrate the bactericidal effect of allicin on water-borne bacteria can be extrapolated from the test results of our laboratory studies on MRSA (30 strains), E. coli, E. Faecalis, F. streptococcus, Candida albicans, Pseudomonas aemginosa, Salmonella typhimurium, Streptococcus pyogenes, B. subtilis, Serratia marcecens contained in our earlier patent application PCT/GB2002/004309.

6. Use of Allicin Against Mites and Bacteria that Destroy Bees.

The Varroa mite is an indigenous parasite of honeybees (including Apis cerana and Apis mellifera). European foul brood disease is caused by a bacterium called Melissococcus plutonius (formerly called Streptococcus plutonius) which invades the mid-gut of four to five day old larvae. It multiplies rapidly in the mid-gut causing death. It only affects larvae in open brood. American foul brood disease is caused by Paenibacillus larvae subsp. Larva which affects the larvae in sealed brood cells. There is also a non notifiable fungal brood disease called chalkbrood Ascophera apis which is a significant problem for some beekeepers.

Test results from our laboratory studies on MRSA (30 strains), E. coli, E. faecalis, Candida albicans, Pseudomonas aeruginosa, Salmonella typhimurium, Streptococcus pyogenes etc; and other studies indicate that the liquid, cream and powder forms of allicin will destroy the Varroa mite, the European foul brood and the American foul brood bacteria.

### Study 1

In this study the anti-microbial activity of allicia (Allisure™ Liquid) was tested against a number of bacterial and fungal pathogens associated with social and solitary bees (Paenibacillus larvae subsp. larvae, Paenibacillus larvae subsp. pulvifaciens, Ascosphaera apis and Ascosphaera aggregata). The minimum inhibitory concentrations (MIC) of allicin were determined using broth microdilution method in the range o£1000 ppm to 0.25 ppm. Allicin liquid showed activity against gram-positive bacterial isolates (NIIC 350 ppm) and fungal isolates (MIC 250 ppm). The anti-microbial activity of allicin was also tested in an agar diffusion test using 250 kg of allicin per disk. Bacterial isolates (P. l. pulvifaciens and P. l. larvae) produced a zone of inhibition in the range of 24-26 mm and 45-50 mm respectively. The fungal isolates produced (A. apis) and 35-37 mm (Aaggregata). The macrolide class antibiotic tylosin (Tylan®50, Elanco Inc, IN) was used as a control in both the MIC assay and in the agar diffusion test. The data from this study points to the potential of allicin to inhibit growth of bee pathogens and prevent occurrence of bee diseases.

We tested allicia (Allisure™ Liquid) activity against several species of entomopathogenic bacteria (Paenibacillus larvae subsp. larvae, Paenibacillus larvae subsp. pulvifaciens) and fungi (Ascosphaera apis and Ascosphaera aggregata) using broth microdilution method to determine minimum inhibitory concentration (MIC) and the agar diffusion test (Kirby-Bauer) to determine zone of inhibition. Bacterial spores were isolated from the diseased honey bee larval samples. A small aliquot of heat-treated bacterial spores was suspended in 100 μl of phosphate buffered saline-pH 7.2 (PBS) and plated on semi-selective J. Agar medium, containing Nalidixic and Pipemidic acid (Alippi, A M (1995) Detection of Bacillus larvae spores in Argentinian honeys by using a semi-selective medium. Microbiologia 1995 11(3): 343-50; and Govan, V A; Allsopp, M H; Davison, S A (1999) PCR Detection Method for Rapid Identification of Paenibacillus larvae. Applied and Environmental Microbiology 65(5): 2243-2245.

The plates were incubated at 33° C. in air containing 6% CO₂ and 95% RH. The initial species identification was based on morphological, biological and cultural characteristics. The bacterial culture was tested for a catalase reaction (Leboffe, M. J and Pierce, B e (1999) A photographic atlas for the microbiology laboratory. Morton Publishing Company. 254 pp). The bacterial colonies were characterized by shape, margins and color. Gram-positive stained smears (Gram-stain Reagents Kit, EMD Chemicals Inc., NJ) were examined for morphological identification of vegetative cells and spores.

DNA based PCR identification was performed to corroborate identification of the bacterial species. Bacterial cells from culture plates were added directly to 30 μl of PCR reaction. The PCR primers used in the reaction were based on the 16S RNA sequence to selectively amplify a fragment of 973 by unique to P. larvae (Govan et al., 1999). The PCR products were visualized by 0.8% agarose gel electrophoresis in TAE buffer and ethidium bromide staining. The reference strains of bacteria were used as a control in PCR reactions and were provided by the National Center for Agricultural Research, Peoria, Ill. P. l. larvae (NRRL B-3560, B-2605) and P. l. palvifaciens (NRRL B-3688, B-3685, B-3689, NRS-1687, P. alvei B383).

Fungal spores (Ascosphaera apis) were collected from black honey bee mummies and (Ascosphaera aggregata) spores were collected from the diseased leaf cutter bees. Bee samples were ground in a tissue homogenizer in PBS, filtered through a coarse membrane and centrifuged 5 min at 12,500 rpm. The concentrated spores were then re-suspended in PBS and stored at 4° C. The aliquots of fungal spores (100 μl of approximately 10⁸-10⁹ spores/ml) were plated on Yeast-glucose-phosphate agar medium (YGPS) containing: yeast extract 1%, KH2PO4 1.35%, soluble starch 1.0%, agar 0.2%, glucose 1.0%, streptomycin sulfate 30.0 μg/ml, ampiciline 50.0 μg/ml (Anderson, D L; Gibbs, A J; Gibson, N L (1998) Identification and phylogeny of spore-cyst fungi (Ascosphaera ssp.) using ribosomal DNA sequences. Mycological Research 102(5): 541-547; and HORNITZKY, M A (2001) Literature review of chalkbrood-a fungal disease of honeybees. A report for the rural industries research and development corporation. New South Wales Agriculture, AU, Publication 01/150, 13 pp) and incubated at 33° C., 6% C02 and 95% RH. The fungal colonies were analyzed by microscopic preparations of aerial mycelia and fungal spore cysts. Identification of fungi species was also confirmed by. PCR analysis. The DNA extraction from fungal mycelia and spores, and the PCR conditions were the same as described by Anderson et al.

Minimum inhibitory concentration (MIC) values were determined for allicin (Allisure™ Liquid) using the broth microdilution method (NORRELL, S A AND MESSLEY, K E (1997) microbiology Laboratory Manual. Principles and applications. Prentice-Hall, Inc. 302 pp) in a range of concentrations from 1000 ppm to 0.25 ppm. Positive controls contained the antibiotic Tylosin (Tylan® 50, Elanco Animal Health Inc., IN) and negative controls contained no antibiotics. Bacterial (P. l. larvae, P. l. pulviphaciens) or fungal (A. apis, A. aggregata) spores (100 μl of approximately 10⁸-10⁹ spores/ml) were added to a 2.5 ml of bacterial or fungal liquid medium containing serial dilutions of allicin. Cultures were incubated in a shaker at 35° C. and 215 rpm. The optical densities of cultures (OD 600) were recorded 24 h and 48 h post inoculation, depending on the growth rate of the microbial species. The MIC values were determined as the lowest concentration of the antibiotic that resulted in the absence of the microbial growth in the culture tube and were replicated three times. Minimum bactericidal concentration values (NBC) were determined for allicin by plating out 100 μl of bacterial cultures derived from the MIC assay. Plates were incubated 24 h and 48 h at 33° C. and 6% CO₂ to observe microbial growth.

### Disk Diffusion Test (Kirby-Bauer)/Zone of Inhibition

Allicin (Allisure™ Liquid) was tested against bacterial and fungal pathogens using a standard disk diffusion method (per Norrell & Messley). The aliquots of bacterial or fungal spores (100 μl of approximately 10⁸-10⁹ spores/ml) were plated on Mueller-Hinton agar medium, 4.0 mm depth. The 6 mm paper discs containing 250 μg of allicin or 5 μg of tylosin (positive control) were placed in the center of each plate. Plates were incubated at 33° C. and 6% CO₂ and a zone of inhibition was measured 24 h, 48 h or 76 h post inoculation depending on the microbial species. All experiments were replicated at least three times.

### Results

The gram-positive bacterial isolates (P. l. pulvifaciens and P. l. larvae) had a MIC value of 350 ppm and fungal isolates (A. apis and A. aggregata) had a MIC value of 250 ppm. Allicin showed only bacteriostatic (no bactericidal) activity against P. l. larvae and P. l. pulvifaciens in the range of 1000 ppm to 25 ppm. The antibiotic tylosin (Tylan®50, Elanco Inc., IN) used as a control and had a very high anti-bacterial activity, a MIC value less than 0.25 ppm. In agar diffusion tests, allicin produced inhibition zones in the range of 24-26 mm for P. l. pulvifaciens and 45-50 mm for P. l. larvae. When tested on the fungal pathogens, allicin produced inhibition zones in the range of 31-35 mm against A. apis and 35-38 mm against A. aggregate. In tylosin controls, P. l. pulvifaciens produced 14-16 mm zone of inhibition. Growth of P. l. larvae was completely inhibited by tylosin. As expected, tylosin failed to inhibit growth of any of the fungal isolates and produced a 0.0 mm zone of inhibition.

### Study 2

### Introduction

There are two serious bacterial diseases of honeybees present in the UK. European foulbrood (EFB) is caused by the bacterium Melissococcus plutonius, although other bacteria, including Paenibacillus alvei and Brevibacillus laterosporus, may also be indicative of the disease. American foulbrood (AFB) is caused by Paenibacillus larvae subsp. larvae, which is usually found in monoculture in infected larvae. EFB can be treated using the antibiotic oxytetracycline in many cases, but colonies with AFB are always destroyed due to the highly infectious nature of the disease. However, the use of antibiotics is not desirable, and it is an aim of the NBU to reduce their use in beekeeping. One way to do this is to investigate other potential treatments. The aim of this study was to assess the effects of a novel product called Allicin, a garlic extract formulation, on bacteria associated with honeybee diseases. The results may indicate if the product is suitable for use as a treatment for foulbrood diseases in the field.

Allicin liquid (nominal concentration=1000 ppm. ai) was obtained from Allicin International Ltd and kept refrigerated.

The bacteria tested were Paenibacillus larvae subsp. larvae, Melissococcus plutonium, Brevibacillus laterosporus and Paenibacillus alvei. All isolates were freshly isolated from diseased material sent to the NBU diagnostic laboratory.

### Culture Media and Incubation Conditions

P. larvae subsp. larvae, B. laterosporus and P. alvei were grown on brain heart infusion plus thiamine (BHIT) agar and broth (SOP NBU/014) under aerobic conditions and M. plutonius was grown on SYPG agar and broth (SOP NBU/015) under anaerobic conditions. All experiments were carried out at 34° C. The concentrations of Allicin liquid investigated were 500, 250, 100, 50, 25 and 10 ppm. Broth was made up at twice the usual concentration, so that when the Allicin-containing component was added, the medium was at the correct strength for bacterial growth. Allicin solution was diluted in sterile deionised water to give the desired concentration when added to autoclaved broth. Controls had an aliquot of sterile deionised water added to them and the final volume for each test culture was 5 ml. Further controls were included, which were the media plus an appropriate volume of Allicin, but not inoculated with bacteria. This would indicate if there were any bacteria present in the test item that may influence the results seen. Both aerobic and anaerobic controls were included.

### Isolation of Bacterial Strains

Bacteria were isolated from diseased samples and subcultured on agar plates until pure, when they were inoculated into broth cultures. Cultures of M. plutonius were isolated anaerobically, then plated out and incubated both aerobically and anaerobically, to confirm that the isolate investigated was this bacterium. A similar microbe, Enterococcus faecalis, can sometimes be isolated from EFB-infected samples and is morphologically difficult to distinguish from M. plutonius. However, the former bacterium grows very well aerobically, whereas M. plutonius is unable to replicate. Thus, if an isolate is able to grow aerobically, it is not M. plutonius. This control mechanism was used throughout the experimental procedures to ensure that the correct organism was tested.

### Inoculation of Test Cultures

Each bacterium was freshly grown in a test tube containing 5 ml broth. A loopful (5 μl) of this bacterial suspension was removed from the culture and inoculated into each test tube according to SOP NBU/131. The same inoculum source for each strain was used for all Allicin dilutions, and all experiments were done in triplicate.

### Confirmation of Results

Where growth occurred in the presence of Allicin, one replicate from each concentration for each bacterium was plated out on the appropriate agar to identify the bacteria that had grown in the broth, to confirm the results (SOP NBU/131). Once growth was evident on this confirmation plate, the culture was assessed with respect to the colony morphology and Gram stained (according to SOP NBU/111) as a further confirmatory test. This is a suitable method as each bacterium tested has distinctive morphology both macro- and microscopically.

Investigation into Bacteriostatic or Bactericidal Effects

Where there was no growth in a replicate, at the end of one week the culture was plated out for single colonies on the appropriate agar. This was to determine whether the test item had bactericidal activity, where all the bacterial cells are killed, or bacteriostatic action, where the cells are unable to replicate in the presence of the substance, but will grow when it is removed. A further test, where 0.5 ml of the broth was transferred into 4.5 ml fresh broth (giving a 1:10 dilution) was also undertaken. There would still be some Allicin in the broth, but it should be present at a low enough concentration for growth not to be affected. Where growth occurred in the absence of Allicin, the broth was examined microscopically and plated out for single colonies to confirm the identity of the bacterium.

### Inhibition of Bacterial Growth by Allicin

Table One summarises the results of the growth inhibition studies.

TABLE Oe
Inhibition of bacterial growth by different
concentrations of Allicin.

| | Growth present at test item | | | | | | |
|---|---|---|---|---|---|---|---|
| | concentration (ppm): | | | | | | |
| Bacterium | 0 | 10 | 25 | 50 | 100 | 250 | 500 |
| M. plutonius | + | + | + | + | +^{a} | − | − |
| P. larvae subsp. larvae | + | + | +^{a} | − | − | − | − |
| P. alvei | + | + | + | + | +^{a} | − | − |
| B. laterosporus | + | + | + | + | +^{a} | − | − |

^{a}weak growth seen, occurred later than lower concentrations

All bacteria grew normally in the absence of the test item, and also appeared normal at the lowest concentrations tested. However, P. larvae subsp. larvae was unable to grow well with Allicin at 25 ppm and growth was completely prevented at concentrations greater than this. The other three bacterial species were able to grow up to and including 100 ppm, although in all three cases, growth was slower and not as strong at the highest concentration. There was no apparent growth of any bacterial strain at 250 ppm or 500 ppm. All three replicates for each concentration and bacterium gave the same results, and the identity of the bacterium was also confirmed successfully in each case.

Investigation into Bacteriostatic or Bactericidal Effects

The results of the investigation into viability of cultures after exposure to Allicin are shown in Table Two:

TABLE TWO
Investigation of growth on agar of cultures that
had not grown in the presence of Allicin.

| | Growth on agar after exposure to test | | | |
|---|---|---|---|---|
| | Item at concentration indicated (ppm) | | | |
| Bacterium | 50 | 100 | 250 | 500 |
| M. plutonius | ND | + | − | − |
| P. larvae subsp. larvae | − | − | − | − |
| P. alvei | ND | + | − | − |
| B. laterosporus | ND | + | − | − |

ND—Not determined

The three species able to grow slightly at 100 ppm were also plated out for the viability study, and all grew well, with no indication that their growth had been compromised by exposure to the test item. However, in every case where there was no growth in the broth culture, there was no growth after the culture had been subcultured onto agar without the addition of Allicin. All cultures were very well mixed before being plated out, but it was possible that the transfer of such a small inoculum decreased the likelihood of capturing viable cells, as there were such low numbers of bacteria in the broth cultures with no growth. It is unlikely that if there were viable cells present in the inoculum they would not have grown. The results of further tests, where a 10% inoculum was transferred to fresh media, are giving in Table Three:

TABLE THREE
Investigation of growth in broth of cultures that
had not grown in the presence of Allicin.

| | Growth in broth after exposure to test | | | |
|---|---|---|---|---|
| | Item at concentration indicated (ppm) | | | |
| Bacterium | 50 | 100 | 250 | 500 |
| M. plutonius | ND | ND | + | + |
| P. Larvae subsp. larvae | * | − | − | − |
| P. alvei | ND | ND | + | + |
| B. laterosporus | ND | ND | + | + |

ND = not determined
* Growth present in one replicate only

For P. alvei and B. laterosporus, Allicin did not kill all bacteria in the cultures as growth occurred after subculture into fresh media. However, a different effect was seen on P. larvae subsp. larvae, as there was growth in just one subculture after a similar transfer. When this isolate was plated out to confirm its identity, it was red in colour, although the colonies looked similar to those usually seen in other respects such as size and colony morphology. It also resembled P. larvae subsp. larvae when examined under the microscope. It is possible therefore that the bacterium had mutated, and this was not a typical result, especially as there was no growth in the other replicates. All three bacteria form spores, a phase in the life cycle of some species of bacteria that enables them to withstand environmental stresses, such as lack of water or nutrients. Many spores have a high resistance to extremes of heat, UV radiation and chemical disinfectants. Cultures of P. alvei and B. laterosporus usually exhibit a high number of spores to vegetative cells, but in P. larvae subsp. larvae cultures, this ratio is considerably lower. Indeed, sporulation in this bacterium may be difficult to achieve in vitro. This may help to interpret these results further, as the two bacteria able to grow well would most likely have had many spores present in the inoculum. These might not germinate in the presence of Allicin (which may have affected the vegetative cells only) but when this stress was removed, i.e. they were subcultured into fresh broth without Allicin, the spores could germinate and growth was seen. It is possible that there were far fewer spores inoculated into the P. larvae subsp. larvae test culture, so this bacterium was not able to survive exposure to the test item.

M. plutonius does not form spores, so any prevention of growth in this experiment will most likely be to any bactericidal effects of Allicin on this bacterium. However, there was growth in the absence of Allicin after exposure, showing bacteriostatic rather than bactericidal effects.

Further work would have to be undertaken to confirm the action of Allicin on these bacteria, including tests that could confirm whether it was sporicidal or affected vegetative cells only. Other work could confirm whether the compound shows bacteriostatic or bactericidal effects, although in the case of P. larvae subsp. larvae, a bactericidal effect appeared to be observed.

7. Effectiveness of Allicin Against Glycopeptide Intermediate Resistant Staphylococcus aureus

Staphylococcus aureus is the most common cause of community- and hospital-acquired infection in many areas of the world. In the 1980s, methicillin-resistant S. aureus (MRSA) emerged and became endemic in many hospitals. Vancomycin was the only antimicrobial agent with effective against some MRSA. In 1996, the first S. aureus strain with decreased susceptibility to vancomycin (glycopeptide intermediate-resistant S. aureus [GISA]) was reported in Japan. By 1997, the first GISA strains were reported in the United States and in 2003 a patient in the UK has died from an infection with a GISA strain. GISA strains can therefore cause serious morbidity and mortality.

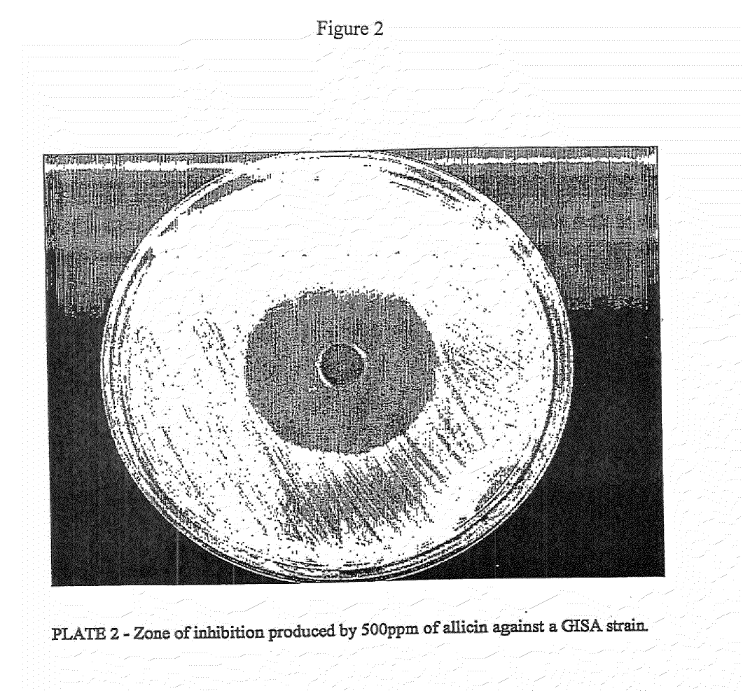

Allicin in a liquid form has been tested against the GISA strain isolated from the UK mortality. In a standard agar diffusion test the strain produced a zone of 37 mm at 500 ppm (FIG. 2) and 30 mm at 300 ppm. The GISA strain was therefore fully susceptible to allicin at our recommended doses for topical use.

### Delivery of Allicin and the Apparatus of WO02/062416

Preparations of allicin and cellulose have been prepared both with and without additional pharmaceutically acceptable excipients. The preparation was delivered to the target areas by the dry spray device of WO02/062416. WO02/062416 describes the use of the apparatus for delivering cellulose to the nasal tract for the treatment of hayfever. This apparatus allows the combination of allicin powder and cellulose to be sprayed by the individual patient onto the target areas (including the nasal tract). In order to test this novel method of delivering allicin to the target areas, mixtures of allicin powder with the cellulose powder provided by the applicant company of WO02/062416, Nasaleze Ltd, were investigated for anti-staphylococcal activity.

The biological activity of allicin against bacteria is well established. In studies contained in our earlier patent application, WO03/024437, we have already shown that certain species of methicillin resistant Staphylococcus aureus (MRSA) are exceptionally susceptible to allicin. Using a susceptible strain of MRSA, we have developed a novel method whereby we can determine whether or not different batches of allicin possess biological activity.

There are a number of tests available to determine the anti-microbial activity of selected agents. Diffusion tests determine the susceptibility of isolates by measuring the zones of inhibition around a measured amount of the anti-microbial agent. Zones of inhibition not more than 6 mm smaller than those of a known control strain indicate that the test bacterium is sensitive to the anti-microbial agent. Zone sizes of 12 mm or less usually indicate antibiotic resistance. There is also an intermediate antibiotic resistant group between with susceptibilities between these levels and zone sizes greater than 12 mm.

### Materials and Methods

Bacteria: MRSA clinical isolate UEL301 was used. Overnight broth cultures in isosensitest broth were prepared.
Media: Isosensitest agar (Oxoid Ltd) were used.
Powders: supplied by Allicin International (cellulose powder from Nasaleze Ltd+allicin powder)

### Method:

A broth containing 10⁵ cfU/ml was prepared in peptone water.
0.2 ml was spread over each isosensitest plate.
Plates were air dried and a 6 mm well cut in the centre of the plate.
A volume of 100 ug or 150 ug of each powder was added to each well.
Plates were incubated overnight at 37 deg C.
The presence of zones of inhibition around a well is indicative of biological activity being present. No zone around the 6 mm well, (as with the negative control) represented no biological activity.

The following ratios of allicin powder and cellulose were used:

### Allicin Powder Cellulose Powder=2:1, 4:1, 6:1 and 8:1.

Tests were also carried out using allicin powder alone, cellulose powder alone and gum acacia powder alone. The concentration of allicin in the allicin powder was nominally 250 ppm.

### Results

COMPARATIVE ZONE SIZES IN MM
(0 represents 6 mm well size)

| Number | Preparation | 100ug | Bioactive | 150ug | Bioactive |
|---|---|---|---|---|---|
| 1 | Negative | 0 (6 mm) | − | 0 (6 mm) | − |
| | control | | | | |
| 2 | Nasaleze | 0 | − | 0 | − |
| | (cellulose | | | | |
| | powder) | | | | |
| 3 | Allicin CPC | 14 | + | 19 | + |
| | 2069/03 | | | | |
| 4 | Allicin CPC | 23 | + | 27 | + |
| | 2102 4-1 | | | | |
| 5 | Allicin CPC | 28 | + | 28 | + |
| | 2102 6-1 | | | | |
| 6 | Allicin CPC | 12 | + | 17 | + |
| | 2069/034-1 | | | | |
| 7 | Allicin CPC | 22 | + | 26 | + |
| | 2102 8-1 | | | | |

The gum acacia alone showed minimal antibacterial activity yielding a zone of 2 or 3 mm. The cellulose powder alone showed no bacterial activity.

Therefore, the above tests demonstrate the antimicrobial activity of a number of allicin/cellulose powder mixtures (delivered by the apparatus of WO021004309 or similar vehicles for delivery of powdered materials) against MRSA and other multiply drug resistant bacteria including MDRTB (Multiply drug resistant tuberculosis), VRSA (Vancomycin resistant Staphylococcus aureus), MRSE (methicillin resistant Staphylococcus epidermidis), PRSP (Penicillin resistant Streptococcus pneumoneae), VRE (Vancomycin resistant enterococci) and VISA (Vancomycin intermediate resistant Staphylococcus aureus).

1.-21. (canceled)

22. A method of disinfecting water comprising adding a composition comprising allicin to the water, to a concentration of allicin in the water of 0.02 to 2.0% (w/v).

23. The method of claim 22, wherein the concentration of allicin in the water is 0.18 to 2.0% (w/v).

24. The method of claim 22, wherein the concentration of allicin in the water is 0.5 to 2.0% (w/v).

25. The method of claim 22, wherein the concentration of allicin in the water is 0.9 to 1.7% (w/v).

26. The method of claim 22, wherein the concentration of allicin in the water is 1.7% (w/v).

27. The method of claim 22, wherein the water is wastewater or storm water or water for washing vegetables or for drinking.

28. The method of claim 22, further comprising contacting the water with allicin for 30 min.

29. The method of claim 28, wherein the contacting is at 37° C.

30. The method of claim 22, wherein the disinfecting further comprises a bacteriocidal effect.

31. The method of claim 22, wherein the composition further comprises a food grade excipient.

32. The method of claim 22, further comprising a second disinfectant.

33. A method for causing a biocidal effect in water comprising adding a composition comprising allicin to the water, to a concentration of allicin in the water of 0.2 to 2.0% (w/v).

34. The method of claim 33, wherein the concentration of allicin in the water is 0.18 to 2.0% (w/v).

35. The method of claim 33, wherein the concentration of allicin in the water is 0.5 to 2.0% (w/v).

36. The method of claim 33, wherein the concentration of allicin in the water is 0.9 to 1.7% (w/v).

37. The method of claim 33, wherein the concentration of allicin in the water is 1.7% (w/v).

38. The method of claim 33, wherein the water is wastewater or storm water or water for washing vegetables or for drinking.

39. The method of claim 33, further comprising contacting the water with allicin for 30 min.

40. The method of claim 39, wherein the contacting is at 37° C.

41. The method of claim 33, wherein the composition further comprises a food grade excipient.
